(19) United States
(12) Patent Application Publication
(10) Pub. No.: US 2003/0074018 A1
(43) Pub. Date: Apr. 17, 2003

(54) Sealing device

(76) Inventors: Jan Torstensen, Virum (DK); Allan Tanghoj, Kokkedal (DK)

Correspondence Address: JACOBSON HOLMAN PLLC, 400 SEVENTH STREET N.W., SUITE 600, WASHINGTON, DC, 20004, US

(21) Appl. No.: 10/058,375

(22) Filed: Jan. 30, 2002

(30) Foreign Application Priority Data
Oct. 12, 2001 (DK) PA 2001 01510

(51) Int. Cl. A61M 29/00

(52) U.S. Cl. 606/197

(57) ABSTRACT

A sealing device for sealing externally debouching, natural or artificial body canals of animals or human beings. The device, made of a resilient material such as compressible open celled foam, enables liquid tight sealing against the inner wall of the body canal. The device may optionally be integrated with or include a sealing element. When used in connection with a bowel system, the device may be an integrated part of an instrument, such as a probe, or may have the form and purpose of a plug.

### BACKGROUND OF THE INVENTION

1. Field of the Invention

The present invention relates to a sealing device for sealing externally debouching, natural or artificial body canals of animals or human beings, the device enabling liquid tight sealing against the inner wall of the bowel system of the animal or human being, especially when performing irrigation through a natural or artificial body opening of the animal or human being. The invention further relates to the use of such a device.

The device is especially suitable as a sealing member incorporated with a disposable trans-anal or trans-stomal irrigation probe.

Faecal continence can be defined as the ability to control defecation, to be able to distinguish flatus from loose respectively formed faeces or to be able to maintain faecal continence even during sleep without use of external aids. In the bowel system the nerves and muscles of the colon, rectum and anus function together in a closely coordinated manner in order to maintain continence. In case of any malfunction e.g. if a person is suffering from some kind of defect in the control of the bowel, this person will very likely be incontinent. The defects may e.g. occur as result of a spinal cord injury, multiple sclerosis (MS), tumours or metastases.

In addition to surgical treatment of faecal incontinence, medication and regulating nutrition, meal times and defecation habits, enemas or irrigation are some of the treatments that have been practised for long time. Enemas or irrigation may also be used by completely healthy people if they for some reason want to avoid toilet visits for a longer period of time.

2. Description of the Related Art

For these purposes several devices have been proposed in the past. In addition to devices especially for use of performing irrigation for treatment of incontinence, other similar devices have been proposed for irrigation, e.g. products developed for administration of barium sulphate enema or the like as part of a radiological examination.

One example of a prior art device is disclosed in U.S. Pat. No. 3,459,175. The device includes an inflatable balloon for giving enemata. The balloon is located on the probe for introduction of irrigation liquid and is to be inflated when positioned in the rectum of the patient close to the anal opening. The ballooned annular element thereby blocks flow from the bowel through anus.

However, enemata probe sealings of the type provided with inflatable balloons inherently possess a number of disadvantages.

To ensure a sufficiently tight sealing to be obtained when placed in the bowel system, the balloon is often inflated to a greater size than necessary thereby exerting an excessive pressure on the bowel wall and even on a larger surface than intended or necessary. This is likely to induce reflectory contraction of the muscles in the bowel and the lower part of colon, resulting in the enemata probe being forced out through the anal opening. Further to the unpleasant leakage, as a result of such premature displacement of the probe with the balloon still being inflated may cause serious injury to the fragile wall of rectum. Also, when expanding in axial direction the balloon may be brought to cover the openings of the probe, thus stopping the intended flow of liquid into the bowel system. Further, there is a risk that a balloon may be overfilled and may result in a rupture of the balloon. Such a situation may likewise cause damage of the fragile wall of the rectum, leakage of the liquid filled content of the colon sigmoideum and rectum and stress of the patient. Also, the balloon may leak which will let the air of the balloon leak out. As a result of this, the device suddenly may fall out of the rectum followed by an unintended leakage of feacal matter. As the users of enemata probes may have no sensory function in the rectum they may not themselves immediately register if the device is falling out. Finally, balloons used in these devices are often made from latex. This may cause problems for patients and others suffering from latex allergy.

When having to perform irrigation on small children a further problem arises because the devices commercially available today are rather large, they therefore seem relatively scaring both for the small children and for their parents.

Another disadvantage in connection with the mentioned products are that they are too expensive to be used as disposable products. According to specialists these products are used several times for the same patient, while being cleaned in-between uses. Apart from being time consuming due to necessary cleaning steps it will be understood that the risk of contaminating the environment during storage of the product in between each use and the risk of infecting the user is much bigger than compared to the risk when using disposable products.

A disposable kit for trans-anal irrigation described in WO 98/23312. The kit comprises a container for irrigation media and is connected to a catheter or probe through which the enemata liquid is administered into the bowel system. The probe is provided with a fixation member intended to ensure fixation of the probe inside the bowel system during inlet of the enemata liquid. The fixation member is made from a compressible material such as a foam or a moulded elastic material. The fixation member is provided in a compressed state and surrounded by a PVA film which will dissolve when getting into contact with humidity. The fixation member is, as an example made of a polyurethane and has an open structure to allow for air passing through during irrigation.

An open structure favourably enhances compressibility of the fixation member but has proved to imply the disadvantage of permeability for fluids during irrigation leading to undesired leakage of irrigation media and liquid bowel contents.

It is an object of the invention to provide a sealing device for providing liquid tight sealing properties in order to overcome the above mentioned disadvantages of sealing devices.

### SUMMARY OF THE INVENTION

The present invention reveals a device for providing liquid tight sealing while avoiding the above mentioned disadvantages.

According to the invention a sealing device is provided, especially a device for sealing externally debouching, natural or artificial body canals of animals or human beings. The device is made from a resilient material e.g. a compressible open celled foam and can optionally be integrated with or include a sealing element.

The invention further relates to the use of a sealing device as a sealing plug providing faecal continence of a body opening. When further incorporated with a probe for administering liquids into the bowel system, use of the device provides security against leakage when performing irrigation through natural or artificial body canals of animals or human beings.

### BRIEF DESCRIPTION OF THE DRAWINGS

The invention is disclosed in further detail with reference to the drawings in which

FIG. 1 illustrates a sealing device according to the invention incorporated with a probe for administration of enemata liquid and placed in the bowel system close to the anal opening of a human being,

FIGS. 2a-e illustrates the principle of alternative combinations of sealing device materials and sealing elements each constituting a sealing device according to the invention, and

FIG. 3 illustrates a sealing device according to the invention.

### DETAILED DESCRIPTION OF THE PRESENT INVENTION

The device can have the form and purpose of a plug, or it can be an integrated part of an instrument for use in the bowel system e.g. an instrument such as a probe for administering liquid into the bowel system. In order to ease insertion of the probe a small diameter thereof is preferred as is a small total diameter of the probe surrounded with a compressed sealing element. This will provide a probe which is easily insertable and simple to use. In use, the cells of the sealing device will tend to take up liquid from the bowel content or from the administered liquid and thereby expand. In both cases use of the device minimises the risk of inducing plug- or probe-ejecting contractions in the bowel system as the pressure exerted by the device, being a swelled type or not, against the wall of the bowel is low because the body of the sealing element is far more compliant as compared to known sealing devices e.g. such as balloon type plugs or probes and does not to the same extend apply a counter pressure against the bowel wall.

A substantially dome-shaped curvature of the device may even enhance its compliability with the wall of the bowel. It is understood that a more or less pronounced inclination of the device can be chosen to optimise the sealing effect, as can the radial size of the device. Further, a number of alternative shapes of sealing devices are suggested in WO 98/23312. In order to obtain a sealing device which in compressed state has as small a radial extension as possible, a thin walled device is preferred, but also more voluminous shapes of the sealing device are relevant, especially when the sealing element is made from very compressible materials. Finally, a collar shaped termination providing a curvature somewhat deviating from the curvature of the body part of the device may be desirable and can even bring about a better sealing effect against the bowel wall.

Further, the soft and compliant feature of the sealing element is mainly provided by the nature and shape of the material it is made from. A sealing device made from open celled foam provides the device with a desired softness and conformability. At the same time the foam is sufficiently compressible to enable the device to be provided for use in a—in order to ease insertion—desirable small and pleasant compressed shape. Examples of materials for such open celled foams are PU, silicone, PVC and PE.

The device is typically provided for use in a compressed state as described above being wrapped in a thin film which dissolves when brought into contact with e.g. bodily humidity i.e. when being placed inside the bowel system whereby expansion of the device is enabled. An example of material for such a thin and dissolvable film is PVA.

Providing the sealing element with the liquid tight feature can be obtained by incorporating into the sealing device a liquid tight foil or layer which prevents passage of bowel liquids through the sealing device. This foil or layer may form the surface on one or both sides of the device, e.g. on the inner surface, facing the content of the bowel system. When the foil or layer form the inner surface of the sealing device, sealing properties of smooth character provided by the compressible material of the device can become fully exploited. On the other hand, when provided as the outer surface of the sealing device facing the bodily opening of the user extraction of the sealing device may become easier due to relatively smooth surface properties provided by the foil or layer. The foil or layer may even be provided between two layers of compressible material inside the device, whereby an increased anchoring of the compressible material is obtained. This could be desirable when the compressible material chosen is of relative incoherent nature. Examples of materials for such foils or layers are e.g. plastics or hydrophobic non-wovens made from materials such as PU, silicone, TPE, latex or polyolefins.

Providing the sealing element with the liquid tight feature can also be obtained by making use of a foam possessing hydrophobic features. Hydrophobicity is obtainable as an inherent feature of the foam itself. Examples of such hydrophobic foams are foams based on PU, Silicone or PVC. A foam being sufficiently hydrophobic is alternatively obtainable by incorporating a hydrophobic agent into the foam during production or by treating the foam with a hydrophobic agent. Applicable hydrophobicity is obtainable by combining strength of hydrophobicity of material or agent with a sufficiently small cell size of the foam. In order to describe a foam material capable of substantially withstanding liquid pressure exerted thereon by liquid or solid contents in the bowel system, combinations of such foam and hydrophobic agent combinations are many. They can e.g. be combinations of foams chosen from the above mentioned foams and hydrophobic agents like silicone surfactants, various soaps or other surface reactants which increase the surface tension of the foam.

It is to be understood that the device can be made from several adjacent layers of e.g. foam of which at least one layer needs to provide the liquid tight properties to the device.

Providing the sealing element with a liquid tight feature can further be obtained by incorporating super-absorbent particles in the foam. One preferred liquid absorbent material is sodium polyacrylate. Alternative absorbents can be chosen from a group comprising inorganic materials, such as gels, or organic compounds, such as a cross linked polymer, or alginates, reticular carboxymethylcelluloses, grafted starches, natural or modified polysaccharides or synthetic derivatives of acrylamides, acrylonitriles or polyacrylates. When being brought into contact with liquids from the bowel system or from administered irrigation or enemata fluids, the super-absorbent particles will swell and expand, thereby closing the cells of the foam to provide a liquid tight barrier. This phenomenon is known to be an undesirable consequence in relation to the use of super-absorbents in the technical field of absorbing articles, where problems arise when so-called gel-blocking occurs in upper layers of absorbing articles (pads, nappies etc.) as the intended distribution of liquid throughout the product is thereby inhibited.

A device according to the invention can be made from several layers of which at least one need to provide the device with the liquid tight quality arising from one or more of the above mentioned ways of providing a such quality.

To ease removal of the device after use it can preferably be provided with an anchored string or similar separate withdrawal means of types known in the art. This especially counts for plug type devices and a number ways of how to anchor such string or similar withdrawal means is described in European patent number EP 0759734 B1. When forming part of a probe the means for withdrawal of the device is advantageously provided by the probe itself.

### DESCRIPTION OF THE PREFERRED EMBODIMENTS

The invention is now explained more in detail with reference to the drawings showing preferred embodiments of the invention.

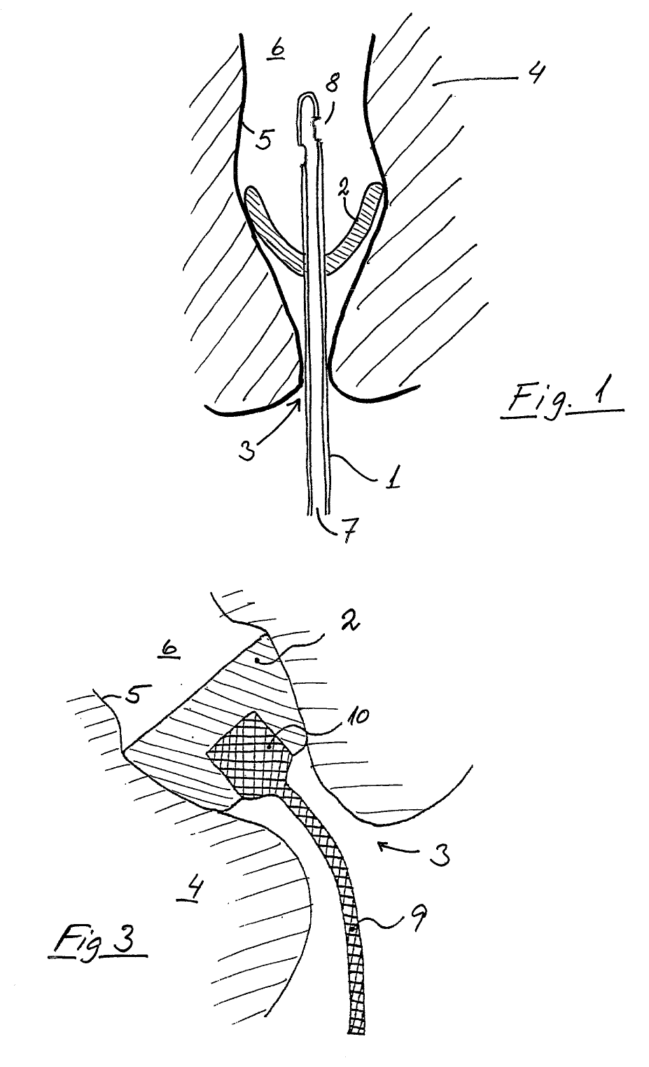

A first embodiment of the invention is illustrated in FIG. 1. A catheter-like probe 1 is shown inserted into the bowel system 6 of a user 4 and positioned close to the anal opening 3. A sealing element 2 sealing against the wall 5 of the bowel system 6 is incorporated with the probe 1 and provides for a liquid tight sealing during introduction of irrigation liquid through the cavity 7 and the openings 8 of the probe 1 out into the cavity of the bowel system 6 of the user 4.

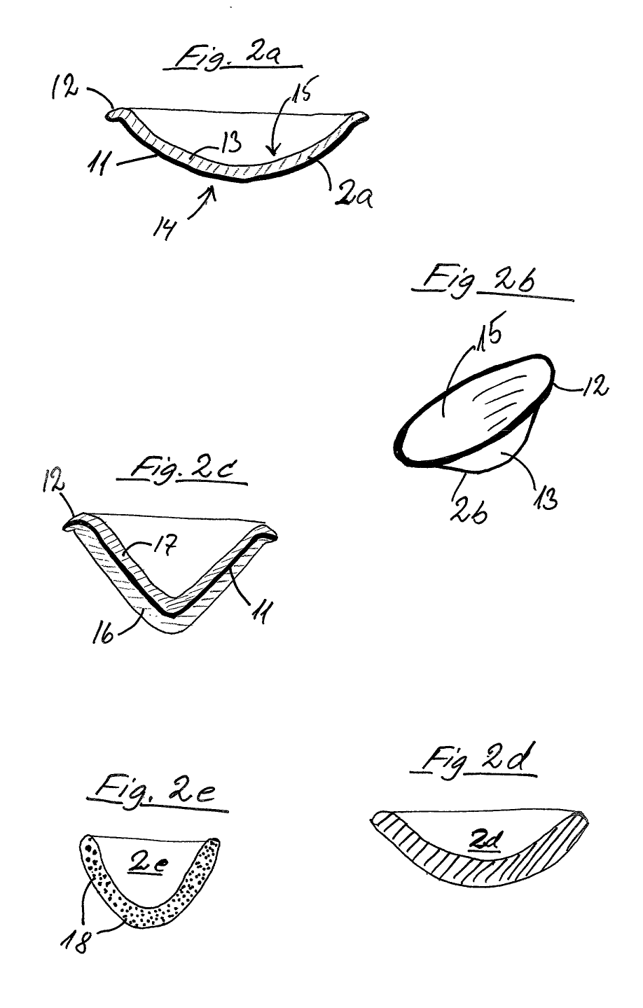

FIG. 2 illustrates sealing devices in a number of preferred alternative shapes as well as it illustrates a number of combinations of sealing device materials and sealing elements.

The outer surface 14 of device 2a illustrated in FIG. 2a is provided by a liquid tight foil layer 11, whereas the device 2b illustrated in FIG. 2b is provided with a liquid tight layer on the inner surface 15. As illustrated in FIG. 2c a liquid tight layer 11 is incorporated between two layers of same or different material 16 and 17 facing the outer surface 14 or the inner surface 15 respectively of device 2c.

FIG. 2d illustrates a device 2d made throughout of a hydrophobic material, while FIG. 2e illustrates a device 2e wherein the sealing element is formed by super-absorbent particles 18 being incorporated in the foam.

Many further combinations of the compressible materials and the liquid tight sealing elements are practicable and may provide for different preferred embodiments for specific uses.

As further illustrated by FIGS. 2a to 2e shape and radial size in relact state, curvature, inclination and thickness of the sealing element as such can vary. Especially a device as illustrated by FIGS. 2a, 2b or 2c provides a collar shaped termination 12 of the device providing a curvature somewhat deviating from the curvature of the body part 13 of the sealing device.

FIG. 3 illustrates a sealing device according to the invention positioned close to the anal opening of a user. The sealing device 2 is provided with withdrawal means 9 through an anchor part 10.

### Methods

In the following and by example only a method for use is described in further detail.

Preferably anus and optionally the anal canal is lubricated with a lubricant such as vaseline to ease insertion of the probe. The probe is inserted through the anal canal and placed in “ampulla recti”, the lower part of the bowel system. A mark or a stop on the probe may be provided to indicates how far the probe it is to be inserted. When inserted the elevated temperature and moisture in the “ampulla recti” will disolve the PVAH layer surrounding the compressed sealing device and the probe will expand. Now the irrigation media—preferably tab water—is pumped from the reservoir to the bowel (in a typical amount of 0,5-2,5 l , preferably 0,75-1 l). When all the desired water has been into pumped to the bowel system the probe is removed immediately or optionally after an additional 1-5 minutes. Evacuation of the bowel will take place over the next maybe 15-45 minutes by reflex action of the bowel system.

1. Sealing device for sealing externally debouching, natural or artificial body canals of animals or human beings, the device enabling liquid tight sealing against the inner wall of the bowel system of the animal or human being, the device further being made from a resilient material and the device optionally being integrated with a sealing element.

2. Sealing device according to claim 1 wherein the liquid tight sealing feature is provided by the resilient material itself.

3. Sealing device according to claim 1 wherein the device is a plug.

4. Sealing device according to claim 1 wherein the device is incorporated with a probe for administering irrigation liquid.

5. Sealing device according to claim 1 wherein the sealing element is formed by a liquid tight foil or layer.

6. Sealing device according to claim 5 wherein the foil is embedded in between layers of the device.

7. Sealing device according to claim 5 wherein the outer surface of the sealing device is formed by the liquid tight foil or layer

8. Sealing device according to claim 5 wherein the inner surface of the sealing device is formed by the liquid tight foil or layer.

9. Sealing device according to claim 1 wherein the sealing element is provided by the foam itself being hydrophobic.

10. Sealing device according to claim 1 wherein the sealing element is formed by super-absorbent particles being incorporated in the foam.

11. Use of a sealing device as described in claim 1 for liquid tight sealing of externally debouching, natural or artificial body canals of animals or human beings.

12. Use of a sealing device as described in claim 1 for liquid tight sealing during administration of liquids through externally debouching, natural or artificial body canals of animals or human beings.
